(19) United States
(12) Patent Application Publication
(10) Pub. No.: US 2011/0201923 A1
(43) Pub. Date: Aug. 18, 2011

(54) METHOD AND SYSTEM OF ELECTROMAGNETIC TRACKING IN A MEDICAL PROCEDURE

(75) Inventor: Eric Shen, Croton on Hudson, NY (US)

(73) Assignee: KONINKLIJKE PHILIPS ELECTRONICS N.V., EINDHOVEN (NL)

(21) Appl. No.: 13/125,994

(22) PCT Filed: Oct. 12, 2009
(86) PCT No.: PCT/IB09/54482

§ 371 Date: Apr. 26, 2011

Related U.S. Application Data
(60) Provisional application No. 61/109,990, filed on Oct. 31, 2008.

(51) Int. Cl. A61B 5/05

(52) U.S. Cl. 600/424

(57) ABSTRACT

A tracking system (300) for a target anatomy of a patient (305) can include a first marker (10) having a size and shape for insertion into the patient to reach the target anatomy where the first marker has a first electromagnetic (EM) sensor (50) and an imageable region (90), a plurality of second markers (310) having a size and shape for adhesion to the patient in proximity to the target anatomy where the second markers each have a second EM sensor and are imageable, a field generator (340) adapted for applying a magnetic field to the target anatomy and inducing a current in the first and second sensors, and a processor (11, 320) having a controller adapted to determine positions of the first and second markers based on the induced currents.

The present application relates to the therapeutic arts, in particular to electromagnetic tracking for medical procedures and will be described with particular reference thereto.

Various techniques and systems have been proposed to improve the accuracy of instrumentality placement (e.g., catheter placement) into tissue based on measurements from 3D imaging formats. These imaging formats attempt to locate a needle entry device in relation to therapy-targeted tissue, such as MRI detected target tissue. These imaging formats generate imaging data that are used to determine the appropriate positioning of the needle during treatment, which needle typically is placed in a guide device and moved into the tissue.

In many cases, the medical device is delivered solely on the basis of this imaging data information and confirmation of the final medical device position relative to the target requires a second set of images to be acquired. In cases where tissue stiffness variations are extreme, the medical device may deviate from the desired path. Similarly, the medical device may distort the tissue itself and thereby move the target tissue to a new location, such that the original targeting coordinates are no longer correct.

Accordingly, there is a need for a technique and system for accurately placing surgical devices in a target anatomy during a medical procedure.

The Summary is provided to comply with U.S. rule 37 C.F.R. §1.73, requiring a summary of the invention briefly indicating the nature and substance of the invention. It is submitted with the understanding that it will not be used to interpret or limit the scope or meaning of the claims.

In accordance with one aspect of the exemplary embodiments, a method of tracking a medical device can include providing at least three markers for registration of an electromagnetic space of a target anatomy with an imaging space of the target anatomy where the markers comprise a first marker and a second marker, positioning the first marker into the target anatomy of a patient where the first marker has a first electromagnetic (EM) sensor and an imageable region, positioning the second marker on the patient in proximity to the target anatomy where the second marker has a second EM sensor and is imageable, inducing a current in the first and second sensors using a field generator external to the patient, determining positions of the first and second markers based on the induced currents, performing imaging of the target anatomy that includes visualization of the imageable region and the second marker, and registering the electromagnetic space of the target anatomy with the imaging space of the target anatomy based at least in part on the determined positions of the first and second markers and the visualization of the imageable region and the second marker.

In accordance with another aspect of the exemplary embodiments, a computer-readable storage medium can include computer-executable code stored therein, where the computer-executable code is configured to cause a computing device, in which the computer-readable storage medium is provided, to execute the steps of obtaining positions of first and second markers based on induced currents in the first and second markers where the first marker is in a target anatomy and the second marker is external to the target anatomy, obtaining imaging of the target anatomy that includes visualization of the second marker and an imageable region associated with the first marker, and registering an electromagnetic space of the target anatomy with an imaging space of the target anatomy based at least in part on the positions of the first and second markers and the visualization of the imageable region and the second marker.

In accordance with another aspect of the exemplary embodiments, a tracking system for a target anatomy of a patient can include a first marker having a size and shape for insertion into the patient to reach the target anatomy where the first marker has a first electromagnetic (EM) sensor and an imageable region, a plurality of second markers having a size and shape for adhesion to the patient in proximity to the target anatomy where the second markers each have a second EM sensor and are imageable, a field generator adapted for applying a magnetic field to the target anatomy and inducing a current in the first and second sensors, and a processor having a controller adapted to determine positions of the first and second markers based on the induced currents.

The exemplary embodiments described herein have a number of advantages over contemporary systems and processes, including accuracy of surgical device placement. Additionally, the system and method described herein can be utilized with existing surgical devices having tracking devices. Still further advantages and benefits will become apparent to those of ordinary skill in the art upon reading and understanding the following detailed description.

The above-described and other features and advantages of the present disclosure will be appreciated and understood by those skilled in the art from the following detailed description, drawings, and appended claims.

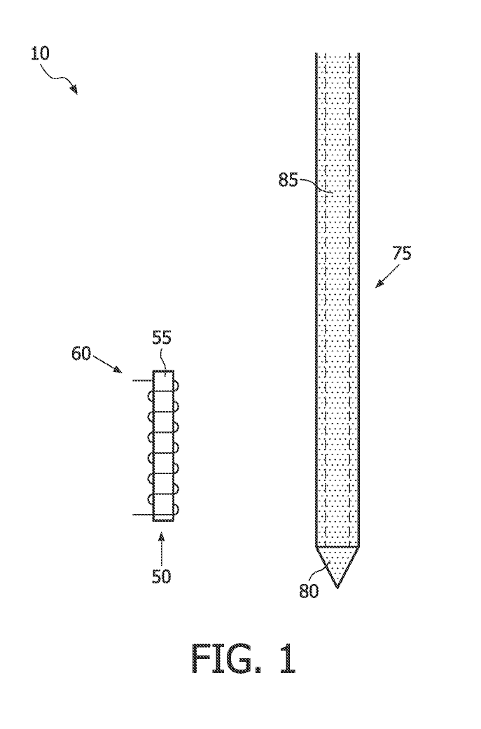

FIG. 1 is a schematic illustration of an exploded view of an internal marker for use with a tracking system of one exemplary embodiment;

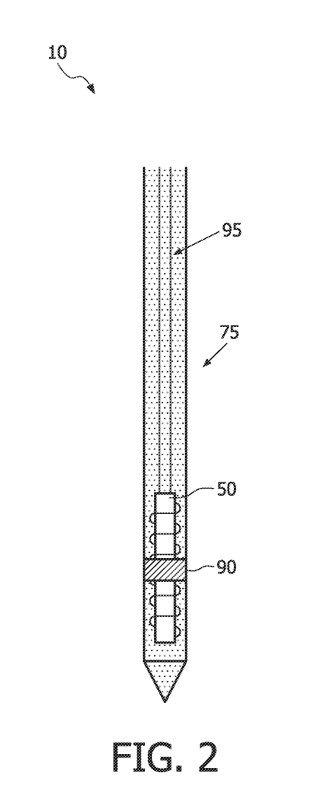

FIG. 2 is another schematic illustration of the internal marker of FIG. 1;

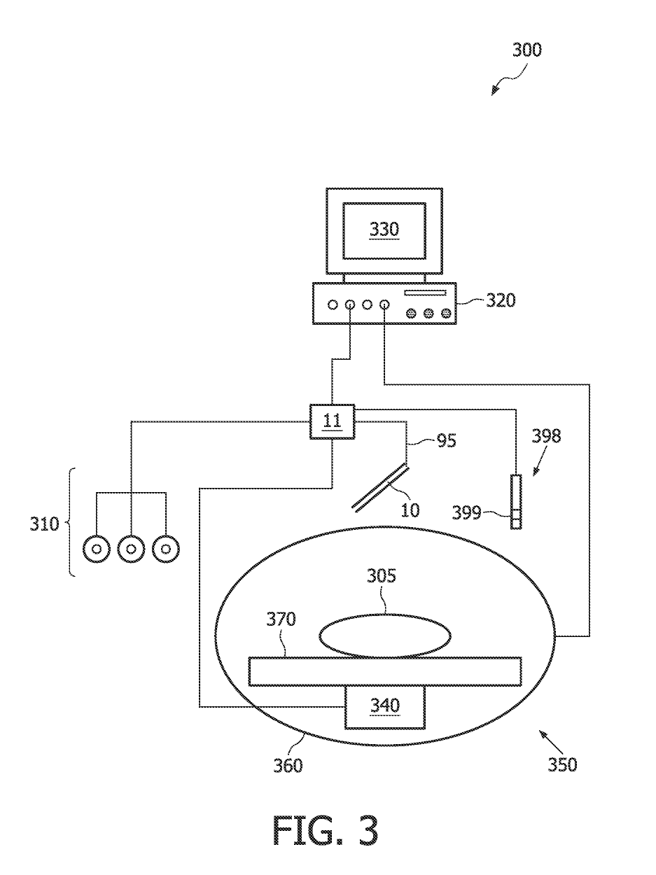

FIG. 3 is a schematic illustration of the tracking system coupled to the internal marker;

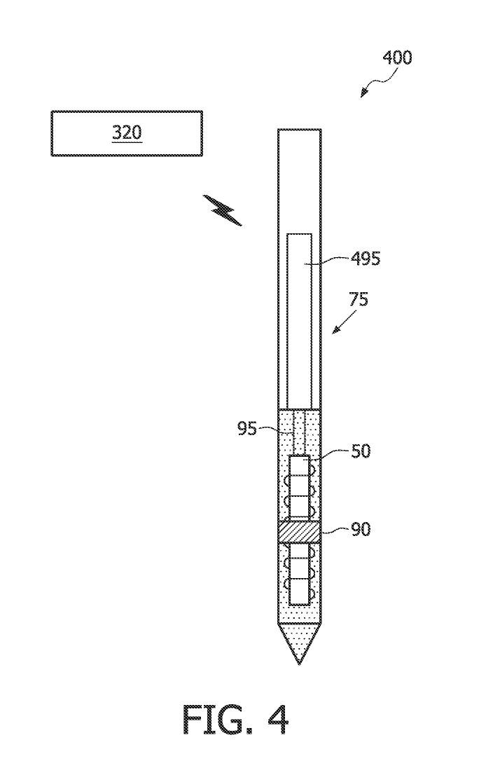

FIG. 4 is a schematic illustration of another exemplary embodiment of an internal marker;

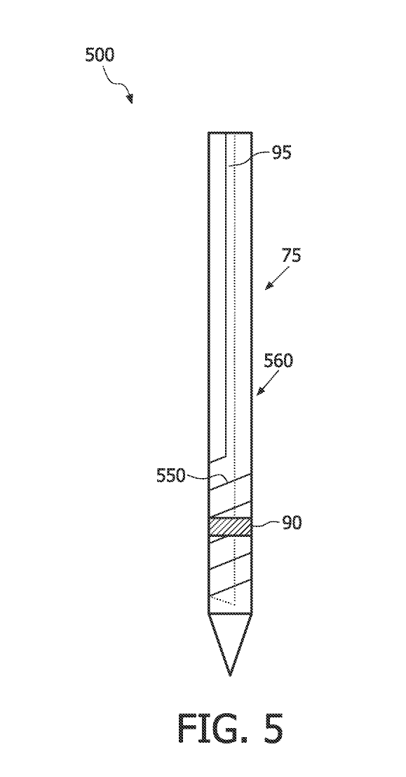

FIG. 5 is a schematic illustration of another exemplary embodiment of an internal marker;

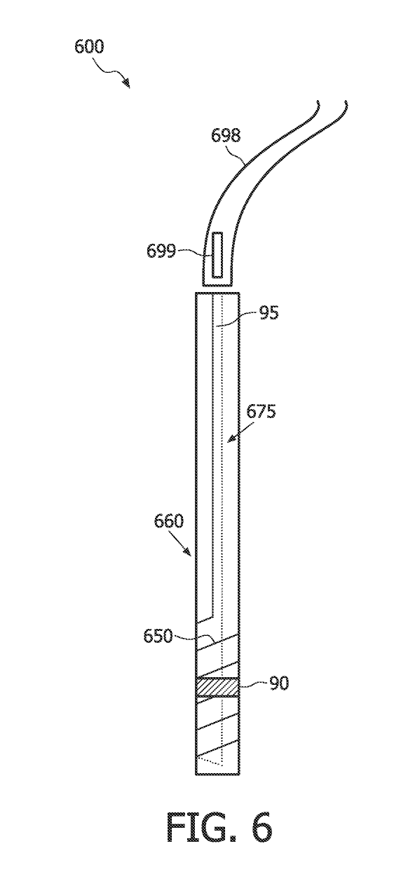

FIG. 6 is a schematic illustration of another exemplary embodiment of an internal marker; and

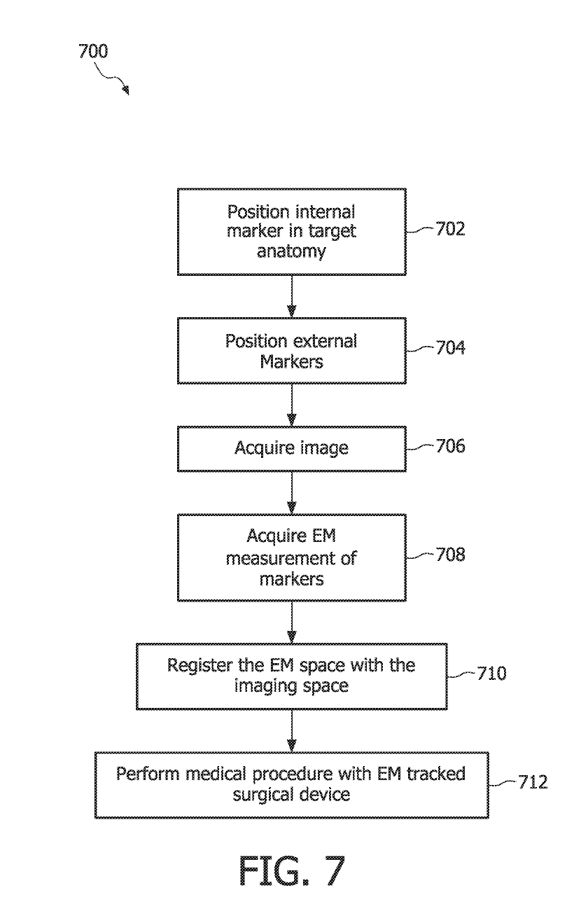

FIG. 7 is a method that can be used by the system of FIGS. 1-6 for performing tracking of a medical device.

The exemplary embodiments of the present disclosure are described with respect to an electromagnetic tracking system for a surgical or other medical device to be utilized during a procedure for a human. It should be understood by one of ordinary skill in the art that the exemplary embodiments of the present disclosure can be applied to, and utilized with, various types of medical or surgical devices, various types of procedures, and various portions of the body, whether human or animal. The use of the method and system of the exemplary embodiments of the present disclosure can be adapted for application to other types of internal markers.

Referring to the drawings, and in particular to FIGS. 1-3, a tracking system 300 can have an internal marker 10 with a sensor device 50. The sensor device 50 can be configured as a sensor coil with a core 55 (e.g., a metal core) and a coiled wire 60 wrapped about the core. The sensor coil 50 can have a size and shape to provide for induction of a current through the wire 60 when the device 50 is exposed to a magnetic field. The particular dimensions of the coil 50, including the diameter and length of the coil and the spacing between the annular portions of the coil can be based on a number of factors, including the strength of the magnetic field, the target anatomy, and/or the presence or potential presence of metal distortions in proximity to the target anatomy. The present disclosure contemplates the use of other coil configurations that allow for induction of a current therein based on exposure to a magnetic field.

In one embodiment, the sensor coil 50 can be inserted into, or otherwise incorporated with, a needle 75 or other device that allows for positioning of the sensor coil within a target anatomy, such as in tissue, adjacent to an organ, and so forth. For example, the needle 75 can have a tapered end 80 for facilitating insertion into a patient to reach the target anatomy, and a channel 85 or other opening along its length for placement of the sensor coil 50 therein. The channel 85 also allows wiring 95 or other connections to connect the sensor coil 50 with an external processor 11 or other computing device.

The needle 75 can have an imaging band 90 or other identification area. The band 90 can be made of a material, and have a size and shape, that allows it to be visible during imaging of the target anatomy. The particular type of material, as well as its size and shape, can be based on a number of factors, including the type of imaging that will be utilized, and the target anatomy that is being imaged. For instance, the band 90 can comprise a gadolinium-doped material where the imaging modality is magnetic resonance imaging. As another example, the band 90 can comprise a plastic or bone-like substance of sufficient density to provide for X-ray attenuation where the imaging modality is computer tomography or X-ray imaging. In one embodiment, the band 90 can be positioned in proximity to the center of the coil 50. The location of the band relative to the sensor coil 50 can be representative of the position and orientation that is determined from the induced current in the sensor coil.

In one embodiment, the tracking system 300 can include a processor 11 that is in communication with the internal marker 10, such as through wires 95 running through the needle 75, as well as a field generator 340 that creates a magnetic field in the target anatomy. The sensor 50 of the internal marker 10 can receive EM signals generated by the field generator 340 which can be positioned in proximity to the patient 305, such as under a bed 370 or other support structure for the patient.

In one embodiment, the field generator 340 can have a plurality of antennas at different orientations. The sensor 50 can pick up the signals from the antennas at various positions and orientations in the target anatomy. From their relative signal characteristics, e.g., relative signal strength, relative phase, etc., the location of the sensor 50 relative to the antennas can be determined.

Tracking system 300 can also include one or more external markers 310, which can be mounted on the patient 305 in proximity to the target anatomy. Each marker 310 can include an electromagnetic sensor unit, such as a sensor coil, which is in communication with the processor 11. The external markers 310 can comprise a material that is visible during imaging. The internal marker 10 and external markers 310 can provide position and orientation information to the processor 11 based on inducing a current in the sensor unit using the field generator 340. The induced current in the markers 10, 310 can be a function of the position and orientation of the sensor 50 relative to the field generator 340. The processor 11 can analyze the current or data representative of the current to make this determination as to position and orientation. As will be described again later, various numbers of the internal markers 10 and the external markers 310 can be utilized by tracking system 300, including three or more markers.

Tracking system 300 can be used with, or can include, an imaging modality 350, such as a high resolution imaging modality, including CT scanner 360. For example, a high resolution image of the target anatomy of patient 305, including the internal marker 10, one or more external markers 310, and the surrounding region (e.g., tissue, organs, vessels, and so forth) can be generated by the CT scanner 360 and stored in a CT image memory. The CT image memory can be incorporated into workstation 320 and/or can be a separate storage and/or processing device. A closed CT scanning device 360 is shown in FIG. 3 for illustrative purposes, but the present disclosure contemplates the use of various imaging devices, including a moving C-arm device or open MRI. The present disclosure contemplates the use of various imaging modalities, alone or in combination, including MRI, ultrasound, X-ray, CT, and so forth. The present disclosure also contemplates the imaging modality 350 being a separate system that is relied upon for gathering of images, including pre-operative and/or intra-operative images.

In one embodiment, the processor 11 can be an EM tracking system processor which receives the sensed current from the sensor coils of a plurality of markers and in combination with information from the EM field generator calculates the position and orientation information for the sensor coil. The processor 11 can then provide the position and orientation information to another process 320 (e.g. a computer workstation). In another embodiment, images from the CT scanner 360 can be provided to or interface directly with the computer workstation 320. Similarly, the position and orientation information from the processor 11 can be provided to the computer workstation 320 in order to guide a medical procedure. The exemplary embodiments describe use of separate processors 11 and 320 for performing signal processing of the sensor current and performing registration. However, the present disclosure contemplates use of a single processor or more than two processors to perform these functions or portions of these functions, such as a computer workstation that receives raw current data from the markers 10, 310 and receives the imaging data from the CT scanner 360, and then performs the registration based at least in part on this information.

The computer workstation 320 can utilize the EM data from markers 10 and 310 for registration of the EM space with the imaging space. The band 90 of internal marker 10 and each of the external markers 310 are visible in the imaging, which allows for various registration techniques to be utilized, including point-by-point registration. For example, the position and orientation of the EM tracked markers 10, 310 and their visibility in the CT image of imaging modality 350 can be used to register the EM measurements to the frame of reference of the CT image.

The resulting registration of the EM space with the imaging space can then be utilized intra-operatively for tracking of surgical device 398 that includes EM sensors 399. The registration can be utilized to transfer the EM measurements of the surgical device sensors 399 from the EM frame of reference to the CT image frame of reference, which can be displayed by display device 330. In one embodiment, the display of the surgical device 398 through use of EM tracking and imaging can be in real-time. In another embodiment, system 300 can register the EM measurements of the markers 10, 310 and/or the surgical device 398 to the frame of reference of the CT image without user intervention. In another embodiment, system 10 can graphically display the EM measured positioning overlaid or super-imposed onto the CT image, such as through use of display 330. In one embodiment, the user can accept, reject, or edit the registered EM measurements of the positions as an accurate registration, and then proceed with the surgical procedure.

The present disclosure contemplates other techniques being utilized in addition to the markers 10, 310. For example, the exemplary embodiments can utilize image correlation or processing algorithms for localization. For instance, one or more features that appear in the image and have a known position can be utilized by the image correlation algorithms, such as portions of the surgical device 398.

In one embodiment, the tracking system 10 can use various tracking components to track surgical device 398, such as those available from Traxtal Inc. or Northern Digital Inc. The tracking of surgical device 398 can be performed using the field generator 340 and the processor 11 or can be performed using other components based on the registration performed by the computer workstation 320.

Referring to FIG. 4, another exemplary embodiment of an internal marker is shown and generally represented by reference numeral 400. The marker 400 can include one or more components described above with respect to marker 10, including the sensor coil 50, the needle 75, and the band 90. Marker 400 can include a controller 495 having a wireless transmitter. The controller 495 can be operably coupled to the sensor coil 50 by wires 95 and can wirelessly transmit positioning data, representative of the induced current in the sensor coil, to a receiver, such as one that is operably connected to the processor 11. In one embodiment, the controller 495 can generate the positioning data from its own analysis of the induced current in the sensor coil 50. The components and techniques used for wirelessly transmitting the positioning data can vary, and can include RF signals. The controller 495 can have its own power source (e.g., a battery) and/or can be a passive device that is powered by an external signal, such as an RF signal or other wireless power field.

Referring to FIG. 5, another internal marker is shown and generally represented by reference numeral 500. The marker 500 can include one or more components described above with respect to marker 10, including the needle 75 and the band 90. Marker 500 can provide for a sensor coil 550 that is formed along the outer surface 560 of the needle, or embedded therein (such as being positioned in a channel or groove formed in the outer surface). The coil 550 can be connected to wires 95 that are also formed along the outer surface 560 of the needle, or embedded therein (such as being positioned in a channel or groove formed in the outer surface), and which can be connected to the processor 11 for providing the induced current thereto. In one embodiment, the coil 550 and/or the wires 560 (or a portion thereof) can be printed along the outer surface 560. In this example, the printed wires can then be connected to insulated wires, such as through soldering, which are connected to the processor 11.

Referring to FIG. 6, a catheter is shown and generally represented by reference numeral 600. The catheter 600 can be a hollow device that allows for passing surgical instruments therethrough, such as surgical device 698. The catheter 600 can include one or more components described above with respect to marker 10, including the band 90. Catheter 600 can provide for a sensor coil 650 that is formed along, or embedded in, the outer surface 660 of the catheter body 675 in proximity to the distal end of the catheter. The coil 650 can be connected to wires 95 that are also formed along, or embedded in, the outer surface 660 of the catheter body 675, and which can be connected to the processor 11 for providing the induced current thereto. In one embodiment, the coil 650 and/or the wires 95 (or a portion thereof) can be printed along the outer surface 660. In this example, the printed wires can then be connected to insulated wires, such as through soldering, which are connected to the processor 11.

The surgical device 698 can include one or more tracking sensors 699, such as a sensor positioned at the tip or distal end of the surgical device, so that the device can be tracked by system 300. In one embodiment, the catheter 600 can be flexible, including use of shape memory alloys. In another embodiment, the catheter 600 can have a non-linear shape with a plurality of sensors coils 650 and bands 90 positioned along the catheter, such as along peaks and valleys of the non-linear length.

Referring additionally to FIG. 7, a method 700 of electromagnetic tracking in a medical procedure is shown. Method 700 can be employed for various types of medical treatments where positioning of a medical device is a desired criteria of the procedure. In step 702, the internal marker 10 can be inserted into the target anatomy. The internal marker 10 can have a size and shape that allows for insertion directly into the target anatomy without the need for facilitating instruments, such as a catheter or the like, although the present disclosure also contemplates the use of such facilitating instruments with the marker 10.

In step 704, one or more external markers 310 can be mounted on the patient 305 in proximity to the targeted anatomy and the implanted internal marker 10. In step 706, a high resolution image of the target anatomy including the internal and external markers 10, 310 and the surrounding tissue can be generated by the imaging device and provided to the computer workstation 320. In step 708, the position and orientation of each of the markers 10, 310 can be obtained using the tracking system through inducing current in each of the markers using the field generator 340, and then analyzing the current, including strength and phase, to determine the position and orientation of the markers, such as through use of processor 11. The processor 11 can then transmit this information to the computer workstation 320.

In step 710, the computer workstation 320 can utilize the EM data from the markers 10, 310, as processed by the processor 11, and in combination with the visual data from the band 90 and the external markers in the CT image, can register the EM space with the imaging space. The registration process can be based on different numbers of the markers, including three or more markers. The three or more markers can be various combinations of internal and external markers 10, 310, including a single internal marker 10 and two or more external markers 310. In step 712 the medical procedure is performed using the EM tracked surgical device.

In one embodiment, the registration process can be a point-to-point registration. Once the registration has occurred, the EM space can then be utilized for tracking the medical device 398 during a medical procedure through use of the EM sensors 399 coupled to the device. In another embodiment, imaging can be obtained during the medical procedure and the EM tracking of the medical device 398 combined with the imaging for display to the clinician. The accuracy of the EM tracking of the medical device 398 can be increased through use of the registration process that utilizes the internal marker 10 and the external markers 310.

The present disclosure can provide an internal active fiducial marker to be used in minimally invasive medical procedures, which has a sensor coil marked so as to be visible in a medical image and which provides position readings in an electromagnetic tracking system space. The internal active fiducial marker can be placed inside a patient's body. The marker can include a sensor coil that is recognized by the electromagnetic tracking system. The central region of the sensor coil can be marked to be visible in the medical image. The active fiducial marker can be integrated into a mechanical tool such that it can be inserted into the body. The mechanical tool can also provide a conduit for sensor coil wires. The electromagnetic tracking system can compute the position of the sensor coil, and from it the position of the medical instrument being tracked by the electromagnetic tracking system. The active fiducial marker can be visible in the image space and can gives position readings in the electromagnetic tracking system space, allowing registration of the two spaces. The active fiducial marker can also compensate for electromagnetic tracking system space position error as a result of EM field distortion caused by metal in or near the electromagnetic tracking system space.

The invention, including the steps of the methodologies described above, can be realized in hardware, software, or a combination of hardware and software. The invention can be realized in a centralized fashion in one computer system, or in a distributed fashion where different elements are spread across several interconnected computer systems. Any kind of computer system or other apparatus adapted for carrying out the methods described herein is suited. A typical combination of hardware and software can be a general purpose computer system with a computer program that, when being loaded and executed, controls the computer system such that it carries out the methods described herein.

The invention, including the steps of the methodologies described above, can be embedded in a computer program product. The computer program product can comprise a computer-readable storage medium in which is embedded a computer program comprising computer-executable code for directing a computing device or computer-based system to perform the various procedures, processes and methods described herein. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. Therefore, it is intended that the disclosure not be limited to the particular embodiment(s) disclosed as the best mode contemplated for carrying out this invention, but that the invention will include all embodiments falling within the scope of the appended claims.

The Abstract of the Disclosure is provided to comply with U.S. rule 37 C.F.R. §1.72(b), requiring an abstract that will allow the reader to quickly ascertain the nature of the technical disclosure. It is submitted with the understanding that it will not be used to interpret or limit the scope or meaning of the claims.

What is claimed is:

1. A method of tracking a medical device, the method comprising:
providing at least three markers for registration of an electromagnetic space of a target anatomy with an imaging space of the target anatomy, the at least three markers comprising a first marker (10) and a second marker (50);
positioning the first marker into the target anatomy of a patient (305), the first marker having a first electromagnetic (EM) sensor (50) and an imageable region (90);
positioning the second marker (310) on the patient in proximity to the target anatomy, the second marker having a second EM sensor and being imageable;
inducing a current in the first and second sensors using a field generator (340) external to the patient;
determining positions of the first and second markers based on the induced currents;
performing imaging of the target anatomy that includes visualization of the imageable region and the second marker; and
registering the electromagnetic space of the target anatomy with the imaging space of the target anatomy based at least in part on the determined positions of the first and second markers and the visualization of the imageable region and the second marker.

2. The method of claim 1, further comprising:
positioning a third marker of the at least three markers on the patient in proximity to the target anatomy, the third marker having a third EM sensor and being imageable;
performing the registration of the electromagnetic space of the target anatomy with the imaging space of the target anatomy based at least in part on the determined positions of the first, second and third markers and the visualization of the imageable region and the second and third markers;
positioning the medical device (398) into the target anatomy; and
tracking positions of the medical device using the field generator (340) and at least one EM sensor (399) connected to the medical device.

3. The method of claim 2, further comprising superimposing the tracked positions of the medical device (398) on the imaging of the target anatomy.

4. The method of claim 3, further comprising displaying the superimposed images in real-time.

5. The method of claim 1, wherein the first marker (10) is a needle having a tapered distal end (80) with a size and shape adapted for insertion through tissue of the patient (305) into the target anatomy.

6. The method of claim 5, wherein the first sensor (50) is a sensor coil positioned in a channel (85) formed in the needle (10).

7. The method of claim 1, further comprising providing the induced current of the first marker (10) to a processor (11) by way of wires (95) extending from a proximal end of the first marker, the processor determining the position of the first marker.

8. The method of claim 1, further comprising performing the imaging using at least one of computed tomography, magnetic resonance imaging, and ultrasound imaging.

9. The method of claim 1, further comprising wirelessly transmitting data representative of the induced current to a processor (11) that is external to the patient (305).

10. A computer-readable storage medium in which computer-executable code is stored, the computer-executable code configured to cause a computing device, in which the computer-readable storage medium is provided, to execute the steps of:
obtaining positions of first and second markers (10, 310) based on induced currents in the first and second markers, the first marker being in a target anatomy and the second marker being external to the target anatomy;
obtaining imaging of the target anatomy that includes visualization of the second marker and an imageable region (90) associated with the first marker; and
registering an electromagnetic space of the target anatomy with an imaging space of the target anatomy based at least in part on the positions of the first and second markers and the visualization of the imageable region and the second marker.

11. The computer-readable storage medium of claim 10, further comprising computer-executable code for causing the computing device to:
obtain a position of a third marker (310) based on an induced current in the third marker, the third marker being in proximity to the target anatomy;
perform the registration of the electromagnetic space of the target anatomy with the imaging space of the target anatomy based at least in part on the positions of the first, second and third markers and the visualization of the imageable region and the second and third markers; and
electromagnetically track a surgical device (398) using the registered electromagnetic and imaging spaces of the target anatomy.

12. The computer-readable storage medium of claim 10, further comprising computer-executable code for causing the computing device to perform metal distortion compensation using the registered electromagnetic and imaging spaces.

13. The computer-readable storage medium of claim 11, further comprising computer-executable code for causing the computing device to wirelessly receive the position of the first marker (10).

14. The computer-readable storage medium of claim 11, further comprising computer-executable code for causing the computing device to display positioning of the surgical device (398) superimposed on the imaging of the target anatomy.

15. The computer-readable storage medium of claim 11, further comprising computer-executable code for causing the computing device to obtain the imaging using at least one of computed tomography, magnetic resonance imaging, and ultrasound imaging.

16. A tracking system (300) for a target anatomy of a patient (305), the system comprising:
a first marker (10) having a size and shape for insertion into the patient to reach the target anatomy, the first marker having a first electromagnetic (EM) sensor (50) and an imageable region (90);
a plurality of second markers (310) having a size and shape for adhesion to the patient in proximity to the target anatomy, the second markers each having a second EM sensor and being imageable;
a field generator (340) adapted for applying a magnetic field to the target anatomy and inducing a current in the first and second sensors; and
a processor (11, 320) having a controller adapted to determine positions of the first and second markers based on the induced currents.

17. The system of claim 16, further comprising another processor (320) having a controller adapted to:
obtain imaging of the target anatomy that includes visualization of the first imageable region and the second markers; and
register an electromagnetic space of the target anatomy with an imaging space of the target anatomy based on the determined positions of the first and second markers (10, 310) and the visualization of the imageable region and the second markers.

18. The system of claim 16, wherein the controller of the processor (11, 320) is adapted to:
obtain imaging of the target anatomy that includes visualization of the first imageable region and the second markers; and
register an electromagnetic space of the target anatomy with an imaging space of the target anatomy based on the determined positions of the first and second markers and the visualization of the imageable region and the second markers.

19. The system of claim 18, further comprising a surgical device (398) having a third sensor (399), wherein the field generator (340) induces a current in the third sensor, and wherein the processor (11, 320) tracks positioning of the surgical device based on the induced current in the third sensor and the registration between the electromagnetic and imaging spaces.

20. The system of claim 19, wherein the surgical device (398) comprises a catheter and the third sensor (399) is positioned at the distal end of the catheter.

21. A first marker (10) for use in a tracking system (300) of a target anatomy of a patient (305), the first marker having a size and shape for insertion into the patient to reach the target anatomy and comprising a first electromagnetic (EM) sensor (50) and an imageable region (90), the tracking system including a plurality of second markers (310) having a size and shape for adhesion to the patient in proximity to the target anatomy, the second markers each having a second EM sensor and being imageable, a field generator (340) adapted for applying a magnetic field to the target anatomy and inducing a current in the first and second sensors; and a processor (11, 320) having a controller adapted to determine positions of the first and second markers based on the induced currents.
